(19) United States
(12) Patent Application Publication
(10) Pub. No.: US 2010/0304366 A1
(43) Pub. Date: Dec. 2, 2010

(54) Methods for Determining Virulence and Invasiveness Among Various Staphylococcus Aureus Strains

(76) Inventor: Ling-Chuan Chen WU, Lawrenceville, NJ (US)

Correspondence Address: Lu & Associates, P.C., P.O. Box 1380, HAVERTOWN, PA, 19083, US

(21) Appl. No.: 12/255,126

(22) Filed: Oct. 21, 2008

Related U.S. Application Data
(60) Provisional application No. 60/987,548, filed on Nov. 13, 2007.

(51) Int. Cl. C12Q 1/68

(52) U.S. Cl. 435/6

(57) ABSTRACT

This invention describes quick diagnostic methods to distinguish CA-MRSA MW2 or CA-MRSA USA300 strains in clinical samples from various sources. Fluorescent labeled primers which amplify 1) two unique agr sequences from each strain, 2) a partial spa gene and 3) a partial pvl gene are packaged into a multiplex PCR kit. A real-time instrumentation to amplify and quantify the PCR amplification products, measured by the change of each of the labeled fluorescent reporter and their thermal melting curve, provides a simple and rapid identification of the most virulent and invasive MRSA strains in the United States.

### CROSS REFERENCE APPLICATION

This application claims priority from U.S. Provisional Patent Application No. 60/987,548 filed on Nov. 13, 2007.

### FIELD OF THE INVENTION

The present invention relates to methods for detecting highly virulent and invasive methicillin-resistent Staphylococcus aureus (hereinafter referred to as MRSA) MW2 and USA300 strains in biological samples. More specifically, this invention provides methods for distinguishing S. aureus strains that harbor various virulent and pathogenic determinants from patients samples using primers and multiplex polymerase chain reaction (PCR) such that the amplification of these determinants represent the most pathogenic strains including MRSA USA300 or MRSA MW2.

### BACKGROUND OF THE INVENTION

S. aureus is a potentially pathogenic bacterium that causes a broad spectrum of conditions in human beings, ranging from carrier state to mild skin diseases and life-threatening invasive infection. The carrier rate of S. aureus world-wide is about 30 percent. In the United States hospitals, it was estimated that approximately 500,000 patients contracted an infection per year. Those who infected with S. aureus stay 3 times longer in the hospital and are 5 times more likely to die comparing to patients who are not infected. Furthermore, S. aureus isolated in the patient's blood stream were associated with 20-40 percent fatality.

The infectivity and the severity of the infection caused by S. aureus are mainly determined by the patient's condition as well as by genetic background of the bacteria. The most pathogenic strains carry Panton-Valentine leukocidin (PVL) to destroy neutrophiles; staphylococcal protein A (SpA) to induce the production of proinflammatory chemokine and cytokine; various superantigens and enterotoxins to trigger an overstimulation of the immune system; and phenol-soluble modulin (PSM) peptides to lyse human neutrophiles (Wang et al., 2007, Nature Medicine 13:1510-1514, published online 11 Nov. 2007). Although all sequenced S. aureus strains contain gene encoded for PSMs, their expression is tightly controlled by the agr quorum-sensing system (Wang et al., 2007, Nature Medicine 13:1510-1514, published online 11 Nov. 2007). S. aureus strains harbors these virulence genes are potentially infectious to healthy people in all age groups without risk factors. Although the majority of cases presented are primary skin infections, these strains are highly contagious. They have the potential to quickly progress to life-threatening infections such as necrotizing pneumonia, meningitis, and sepsis in healthy, young, immuno-competent individuals.

The new emerging community-acquired methicillin-resistant S. aureus (hereinafter referred to as CA-MRSA) that derived from the acquisition of methicillin-resistant gene cassettes (SCCmec) VI or V in a virulent S. aureus background has become the most important public health threat since early twenties. Outbreaks in households, day-care center, school and prison have been reported in recent years. It affects a large population in a very short period of time, and accounted for 98 percent of overall S. aureus infections currently. With the growing colonization rate of 0.8 percent in 2001 to 9.5 percent in 2005, CA-MRSA is the most common pathogen cultured in ER skin infections now. Approximately 50 percent of ER visit with skin problems are due to CA-MRSA, while secondary necrotizing pneumonia caused by CA-MRSA had also been reported in the 2006 flu season.

In contrast to the virulent S. aureus, the so-called hospital-acquired methicillin-resistant S. aureus (HA-MRSA) do not carry the above mentioned virulence factors. The HA-MRSA that established and circulated in most of the US hospitals for the past ten to twenty years affected peoples with risk factors, such as prolong hospital stay, transplantation, catheterization, surgery, antibiotic exposure, etc. These strains rarely infect health care professionals whom, however, can be the carriers that transmit the bacteria from patient to patient. Without the virulence genes, the HA-MRSA enters the human's body and becomes systemic infections through the cuts, wounds, and the inserted catheter tubes and devices. The affected groups are often elderly people, especially those living in nursing home and long-term care facilities. The major problem associated with HA-MRSA is the limited treatment options owing to the existence of the methicillin-resistant gene cassettes carried by HA-MRSA (SCCmec I-III) which also carry multiple non-beta-lactam antibiotic-resistant determinants. For HA-MRSA, the only remaining antibiotics are vancomycin, linezolid, quinupristin and dalfopristin.

Unlike HA-MRSA isolates, CA-MRSA isolates from patients without known MRSA risk factors are generally resistant to fewer non-beta-lactam antibiotics. They grow significantly faster than the nosocomial strains and can be highly virulent to cause serious and often fatal disease in otherwise immunocompetent individuals. The evolution of CA-MRSA is believed to be a recent event due to the acquisition of a novel SCCmec IV (or V) cassette with methicillin-resistant gene by an otherwise susceptible S. aureus. Unlike SCCmec I-III, the characteristic cassettes carried by CA-MRSA, SCCmec IV (or V) is smaller in size and do not carry multiple non-beta-lactam antibiotic-resistant determinants. Depending on the severity of the infection and the local antibiogram, the physicians have choices of antibiotics, including clindamycin, trimethoprim-sulfamethoxazole, doxycycline, minocycline, and rifampin, while the vancomycin and other newer antibiotics can be preserved as the last resort for treating CA-MRSA.

Various SCCmec typing methods that based on amplifying different sequences of MRSA have been developed previously. For example, the amplification of SCCmec right extremity junction sequences for the types i, ii, iii, iv, v, vii (Huletsky et al., 2004, J. Clin. Microbiol. 42:1875-1884) and types xi, xii, xiii, xiv, xv, xvi, xvii, xviii, xix, and xx (Huletsky et al., 2007, United State Patent Application NO. 20070082340); and the amplification of mecA internal sequence and a chromosomal DNA surrounding the integrated mecA for differentiating MRSA and methicillin-resistant coagulase-negative staphylococci (Hiramatsu et al., 2000, U.S. Pat. No. 6,156,507). Moreover, Matsunaga et al. described a method and kit that includes amplifying both mecA and spa genes to distinguish MRSA from methicillin-resistant coagulase-negative staphylococci (Matsunaga et al., 1997, U.S. Pat. No. 5,702,895). However, none of these PCR amplification typing methods can indicate the invasiveness and infectivity of a MRSA strain.

MRSA is conventionally identified by culturing bacteria isolated from clinical specimens and checking for the presence for drug sensitivity in most of the hospital or clinical laboratory in the United States. It is time consuming, requiring one day for cultivation and another day for drug sensitivity testing. Furthermore, the tests do not offer any information about the infectivity or the invasiveness of the strains. The time gap plus the lack of virulence indicators will put the decision making, such as whether to promptly triage the infected patient in isolation, on hold and uncertain. A quick test that can be done on site when receiving a patient in a hospital/long term care/nursing home/prison will be greatly helpful and cost-effective, if the test can also indicate the invasiveness of the strain. Depending on test results, the affected patient can be promptly confined to an isolation room as needed and a highly stringent infection control measure can be applied to prevent further spread of MRSA to other people. In addition, the virulence indicators are very useful in predicting the disease progression, the outcome, and the infectivity to other healthy person. The positive test results justify the need of an aggressive antibiotic treatment followed by continuing monitoring the treatment response and the total eradication of the infection.

To enable a timely response, I invented an alternative method which applies a multiplex PCR coupled to a real-time quantitation of PCR products to indicate the presence of CA-MRSA virulence genes. This invention choose 1) a proper agr gene in either CA-MRSA MW2 or CA-MRSA USA300 strains, 2) pvl gene, and 3) spa gene as target sequences for multiplex PCR amplification products.

The agr gene locus, which comprises two divergent operons from promoters P2 and P3, was initially described in S. aureus as an element controlling the production of exoproteins implicated in virulence. The P2 operon includes four genes, of which two encode elements of density-sensing cassette, agrD encodes the precursor of the autoinducing peptide (AIP), and agrB, whose product is probably involved in processing and/or secretion of AIP (Dufour et al., 2002, J. Bact. 184:1180-1186). The two component sensory transduction system is comprised of AgrC, the membrane sensor, and AgrA, the response regulator. In brief, the AIP derived from AgrD by the action of AgrB interacts with AgrC in the membrane to activate AgrA, which upregulates transcription both from P2, amplifying the response, and from P3, initiating the production of a novel effector: RNAIII. In S. aureus, delta-hemolysin is the only translation product of RNA III (Dufour et al., J. Bact. 2002, 184:1180-1186).

The agr gene locus in S. aureus has been shown to be polymorphic. Sequence variation is particularly marked in the AIP precursor, AgrD; the C-terminal two-thirds of AgrB, and a portion of the N-terminal of AgrC (Ji et al., 1997, Sci. 276:2027-2030). Moreover, Jarraud et al. (Jarraud et al., 2002, Infection and Immunity 70:631-641) had categorized agr alleles into four groups, base on the observation of a relationship between genetic background, agr groups and human S. aureus disease presentation. Thus, the agr group genes are selected from the pathogenic US CA-MRSA strains MW2 (USA400) (Baba et al., 2002, Lancet 359:1819-1827) and LAC (USA300) (Diep et al., 2006, Lancet 367: 731-739) in this invention.

Although the PSM genes are present in all sequenced S. aureus strains, Wang, et al. (Wang et al., 2007, Nature Medicine 13:1510-1514, published online 11 Nov. 2007) detected mush higher in vitro PSM production in the most prevalent CA-MRSA, including MW2 and USA300, when compared to HA-MRSA. Furthermore, all S. aureus PSMs are tightly controlled by the agr quorum-sensing system, and the strain-to-strain differences in PSM production seem to be caused in part by differential agr activity.

The pvl gene encodes PVL which is the major virulence factors in CA-MRSA and certain MSSA strain. The pvl locus encodes two exotoxins—LukS-PV and LukF-PV which act together as subunits, and form a ring with a central pore in the membrane of white blood cells and thus destroy neutrophiles. Although, the predominant CA-MRSA studied so far in the United States has shown a strong association with PVL positively, a recent investigation in Ireland reveled that pvl gene is a poor marker for CA-MRSA, and the presence of pvl gene cannot be used as a sole marker for CA-MRSA (Rossney et al., 2007, J. Clin. Microbiol. 45: 2554-2563).

The spa gene encodes the SpA which is only presented in all S. aureus , but not in Staphylococcus epidermidis or other coagulase-negative Staphylococcus. PVL increases the expression of SpA, which, in turn, binds to tumor necrosis factor receptor and triggers the overproduction of proinflammatory chemokine and cytokine Strains that are spa and pvl positive are most virulent and often associate with fatal necrotizing pneumonia.

This invention utilizes an extensive DNA databases and queries collected and provided by the National Center for Biotechnology Information (hereinafter referred to as NCBI), National Institute of Health, to search unique and non-homologous agr DNA sequences in either CA-MRSA MW2 or CA-MRSA USA300 strains, such that the amplification of these regions represent only CA-MRSA MW2 or CA-MRSA USA300 strains. The amplification of these unique and non-homologous agr DNA sequences, together with the additional spa and pvl genes in one multiplex PCR amplification provides a quick test with the discriminatory power to identify the most predominant, virulent and invasive CA-MRSA in the United States.

### BRIEF SUMMARY OF THE INVENTION

This invention describes quick diagnostic methods to identify the presence of proper agr genetic background in CA-MRSA MW2 or CA-MRSA USA300 strains. Fluorescent labeled primers which are complementary to 1) two unique agr sequences from each strain, 2) a partial spa gene and 3) a partial pvl gene are packaged into a multiplex PCR amplification kit. A real-time instrumentation to quantify the PCR products, measured by the changes in each of the labeled fluorescent reporter and their thermal melting curve, provides a simple and rapid identification of the most virulent/invasive MRSA strains in the United States.

### BRIEF DESCRIPTION OF THE DRAWING

FIG. 1 depicts the flow chart of searching and selecting unique agr gene sequences in CA-MRSA MW2 or USA300 strains for PCR amplification and the designing and labeling of the primers for amplifying these unique agr sequences.

FIG. 2 depicts the flow chart of searching and selecting pvl gene sequences in CA-MRSA MW2 or USA300 strains for PCR amplification and the designing and labeling of the primers for amplifying these pvl sequences.

FIG. 3 depicts the flow chart of searching and selecting spa gene sequences in CA-MRSA MW2 or USA300 strains for PCR amplification and the designing and labeling of the primers for amplifying these spa sequences.

FIG. 4 depicts the overall flow chart of the present invention.

### DETAILED DESCRIPTION OF THE INVENTION

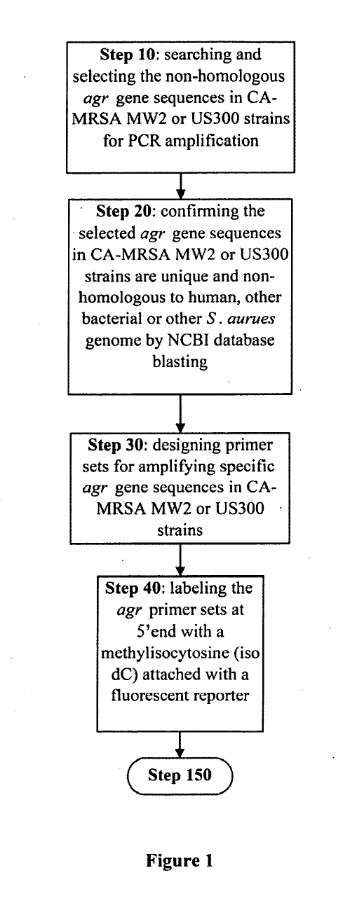

In the first dimension, this invention provides unique agr gene sequences that can represent only CA-MRSA MW2 or USA300 strains. The designing and labeling of the proper primer sequences to amplify these agr gene sequences serve as identifiers to discriminate other staphylococci in clinical samples. A flow chart of steps that describe the invention of proper agr gene and primer sequences is depicted in FIG. 1. Detailed description of FIG. 1 is presented by steps as follows.

FIG. 1 Step 10: Searching and selecting the non-homologous agr gene sequences in CA-MRSA MW2 or USA300 strains for multiplex PCR amplification

This invention utilizes DNA databases and queries collected and provided by NCBI, National Institute of Health, to search the whole genome of CA-MRSA MW2 strain. The search result yields one GenBank accession number BA000033, submitted by Baba et al. (Baba et al., 2002, Lancet 359:1819-1827). A 2760 base pair fragment, spanning agr locus (base pair number from 2108049 to 211088 (GenBank accession number NC_{—}003923.1) in CA-MRSA MW2 strain) was selected for aligning agr locus from CA-MRSA USA300 (GenBank accession number NC_{—}007793.1) and four agr alleles published by Jarraud et al. (Jarraud et al., 2002, Infection and Immunity 70:631-641). Each of the four agr alleles was designated agr-1ₛₐ (GenBank accession number X52543); agr-2ₛₐ (GenBank accession number AF001782); agr-3ₛₐ (GenBank accession number AF001783), and agr-4ₛₐ (GenBank accession number AF288215) (Jarraud et al., 2002, Infection and Immunity 70:631-641). The alignment was carried out by NCBI Blast 2 software (version LASTN 2.2.17 [Aug. 26, 2007]).

A 64 base pair DNA sequence located in agrC (from base pair number 2108961 to 2109024, GenBank accession number BA000033, SEQ ID NO:1) in CA-MRSA MW2 genome was found that is unique from the corresponding agrC sequence derived from each of the CA-MRSA USA300, agr-1ₛₐ, agr-2ₛₐ, agr-3ₛₐ, and agr-4ₛₐ. Similarly, a 142 base pair DNA sequence also located in agrC (from base pair number 2108819 to 2108960, GenBank accession number BA000033, SEQ ID NO:2) in CA-MRSA MW2 genome was also found which was non-homologous to the corresponding agrC sequence derived from each of the CA-MRSA USA300, agr-1ₛₐ, agr-2ₛₐ, and agr-4ₛₐ. However, they are highly similar with the corresponding agr-3ₛₐ sequence, with only 2 mis-matched base pairs.

A similar search was carried out using a 5001 base pair sequence spanning the agr locus in CA-MRSA USA300 strain (from base pair number 2145000 to 2150000, GenBank accession number NC_{—}007793.1) to align each of the agr locus in the CA-MRSA MW2, agr-1ₛₐ, agr-2ₛₐ, agr-3ₛₐ, and agr-4ₛₐ. Each of the four agr alleles was designated agr-1ₛₐ (GenBank accession number X52543); agr-2ₛₐ (GenBank accession number AF001782); agr-3ₛₐ (GenBank accession number AF001783), and agr-4ₛₐ (GenBank accession number AF288215) (Jarraud et al., 2002, Infection and Immunity 70:631-641). The alignment was carried out by NCBI Blast 2 software (version LASTN 2.2.17 [Aug. 26, 2007]).

A 65 base pair DNA sequence located in agrB (from base pair number 2147214 to 2147278, GenBank accession number NC_{—}007793.1, SEQ ID NO:3) in CA-MRSA USA300 genome was found that is unique from the corresponding agrB sequence derived from each of the CA-MRSA MW2, agr-1ₛₐ, agr-2ₛₐ, agr-3ₛₐ, and agr-4ₛₐ. Similarly, a 66 base pair DNA sequence located in agrC (from base pair number 2148216 to 2148281, GenBank accession number NC_{—}007793.1, SEQ ID NO:4) in CA-MRSA USA300 genome was found which was non-homologous to the corresponding agrC sequence derived from each of the CA-MRSA MW2, agr-1ₛₐ, agr-2ₛₐ, agr-3ₛₐ, and agr-4ₛₐ.

FIG. 1 Step 20: Confirming the selected agr gene sequences in CA-MRSA MW2 or CA-MRSA USA300 strains are unique and non-homologous to human, other bacterial or other S. aureus genome by NCBI database blasting

To confirm that each of the SEQ ID NO:1 to ID NO:4 is unique and therefore can be used in the PCR amplification products as diagnostic sequences among mixed specimens from human clinical samples, a confirmation test was performed. The confirmation test uses each of SEQ ID NO:1 to ID NO:4 as a query sequence in search for a homologous sequence among databases collected by NCBI GenBank in five categories: a) human genome sequence (Posted date: Apr. 16, 2008 7:40 PM); b) patent sequence (nucleotide sequences derived from the Patent Division of GenBank, Posted date: Aug. 3, 2008 4:57 AM); c) NCBI chromosome sequences (Posted date: Aug. 5, 2008 4:57 AM); d) NCBI chromosome sequence, under bacteria subdivision (Taxid: 2) (Posted date Posted date: Aug. 5, 2008 4:57 AM); and e) NCBI chromosome sequence, under Staphylococcus (Taxid: 1279) (Posted date: Aug. 5, 2008 4:57 AM). The NCBI BLAST Basic Local Alignment Search Tool (nucleotide Blast) was used to test the sequence similarities under three different conditions: 1) highly similar sequence; 2) more dissimilar sequence; and 3) somewhat similar sequence.

The sequence comparison results showed that there is no homology in either SEQ ID NO:1, SEQ ID NO:3, or SEQ ID NO:4 in any of aforementioned five DNA databases. No significant similarity was found even when the least stringent condition—somewhat similar sequence—was chosen as comparison criteria.

The blast result of SEQ ID NO:2 (from base pair number 2108819 to 2108960, GenBank accession number BA000033) against each of the aforementioned five DNA databases shows various hits according to the stringency of the condition (Table 1). The numbers in Table 1 indicate the blast hits.

TABLE 1
Result of blast hits of SEQ ID NO: 2 against various DNA databases

| | | | Somewhat |
|---|---|---|---|
| Database | Highly similar | More dissimilar | similar |
| Human genome | 0 | 0 | 58 |
| Patent sequence | 0 | 0 | 101 |
| NCBI chromosome | 3 (all are | 9 (three are | 147 |
| | Staphylococcus) | Staphylococcus) | |
| NCBI chromosome | 3 (all are | 3 (all are | 41 |
| (Bacteria, Taxid: 2) | Staphylococcus) | Staphylococcus) | |
| NCBI chromosome | 3 (all are | 3 (all are | 84 |
| (Staphylococcus, | Staphylococcus) | Staphylococcus) | |
| Taxid: 1279) | | | |

Various alignments based on the stringency of the condition are described below:

1) Alignment under high stringency condition (highly similar):

SEQ ID NO:2 (from base pair number 2108819 to 2108960, GenBank accession number BA000033) does not align with any of sequences within human or patent sequence databases under most stringent condition (Table 1).

Results shown in Table 1 confirmed that SEQ ID NO:2 (from base pair number 2108819 to 2108960, GenBank accession number BA000033) is identical to the agrC corresponding sequence in another CA-MRSA MW2 strain (NC_{—}003923.1). This region is also 100% identical to an autoinducer sensor protein in a S. aureus strain MSSA476 (from base pair number 2087944 to 2088085, GenBank accession number BX571857.1). MSSA476 also caused severe invasive disease in the community and belonged to a major clone associated with community-acquired diseases that also contains the CA-MRSA strain MW2 (Enright et al., 2000, J. Clin. Microbiol. 38:1008-1015; Holden et al., 2004, PNAS 101:9786-9791).

Result shown in Table 1 also confirmed that SEQ ID NO:2 (from base pair number 2108819 to 2108960, GenBank accession number BA000033) is 98% identical to an autoinducer sensor protein in a S. aureus strain MRSA252 (from base pair number 2184837 to 2184978, GenBank accession number BX571856.1). MRSA252 belongs to the clinically important EMRSA-16 clone that is responsible for half of all MRSA infection in the U.K. (Johnson et al., 2001, J. Antimicrobiol. Chemother. 48:143-144), and is one of the major MRSA clones found in the U.S. (USA200) (McDougal et al., 2003, J. Clin. Microbiol. 41:5113-5120). Two mismatches found in this region occurred at the exact position when aligned to agr-3ₛₐ (GenBank accession number AF001783) corresponding sequence.

2) Alignment under less stringent condition (more dissimilar):

SEQ ID NO:2 (from base pair number 2108819 to 2108960, GenBank accession number BA000033) do not align with any of sequences within human or patent sequence databases under less stringent condition (Table 1).

However, in addition to the homology with three S. aureus strains described above, alignment of SEQ ID NO:2 (from base pair number 2108819 to 2108960, GenBank accession number BA000033) against NCBI chromosome database picked up six sequences which spans SEQ ID NO:2 DNA region from 2108821 to 2108880 under less stringent condition. These six sequences consists of three sequences derived from Homo sapiens chromosome 11 (GenBank accession numbers AC_{—}000143.1, AC_{—}000054.1, and NC_{—}000011.8) with 78% homology; one sequence derived from Pan troglodytes chromosome 11 (GenBank accession numbers NC_{—}006478.2) with 78% homology; and two sequences derived from Rattus norvegicus chromosome 17 (GenBank accession numbers AC_{—}000085.1 and NC_{—}005112.2) with 84% homology.

3) Alignment under the least stringent condition (somewhat similar):

a) Search in human genome database:

A total of 58 blast hits resulted when using SEQ ID NO:2 (from base pair number 2108819 to 2108960, GenBank accession number BA000033) as the query sequence to align human genome database under the least stringent condition (somewhat similar) (Table 1). The distribution of this 58 blast hits clusters mostly within two regions of SEQ ID NO:2—from base pair number 2108820 to base pair number 2108860 and from base pair number 2108898 to base pair number 2108949. The most non-homologous region within SEQ ID NO:2 located at two regions—from base pair number 2108861 to base pair number 2108897 and from base pair number 2108950 to base pair number 2108960.

b) Search in patent sequence database:

A total of 101 blast hits resulted when using SEQ ID NO:2 (from base pair number 2108819 to 2108960, GenBank accession number BA000033) as the query sequence to align database in Patent Division of NCBI GenBank under the least stringent condition (somewhat similar) (Table 1). These blast hits aligned with DNA sequences that are random and short, and except one house mouse gene (GenBank accession number CS788875, 91% homology, spanning the region of SEQ ID NO:2 from base pair number 2108927 to base pair number 2108950), there are no sequence aligned beyond base pair number 2108947 in SEQ ID NO:2. The house mouse gene is unlikely to be mixed with human clinical samples designed to be tested in this invention. The most non-homologous region within SEQ ID NO:2 located at a region spanning base pair number 2108848 to base pair base pair number 2108960.

However, an artificial sequence of forward primer for a DNA macro-array analysis for agrC-MW2 gene was found in this alignment. This artificial sequence (GenBank accession number CS107636 and CS116412) spans the region corresponding to SEQ ID NO:2 from base pair number 2108928 to base pair number 2108947 with 100% homology. This region is not within the range for primer sequence design in this invention.

c) Search in NCBI chromosome database:

A total of 147 blast hits resulted when using SEQ ID NO:2 (from base pair number 2108819 to 2108960, GenBank accession number BA000033) as the query sequence to align NCBI chromosome database under the least stringent condition (somewhat similar) (Table 1). Excluding the homology in the three S. aureus strains, the distribution of the rest of 144 blast hits are random but there are only 6 hits with its 3′ end extended to the region corresponding to SEQ ID NO:2 base pair number 2108960. However, these 6 hits include two genes in mouse genome (GenBank accession number NC_{—}000078.5 and AC_{—}000034.1); one Equus caballus gene (GenBank accession number NC_{—}009148.2); two Rattus norvegicus genes (GenBank accession number NC_{—}005101.2 and AC_{—}000070.1); and one Canis familiaris gene (GenBank accession number NC_{—}006620.2). Those genes are unlikely to be mixed with human clinical samples designed to be tested in this invention. The most non-homologous region within SEQ ID NO:2 located at a region from base pair number 2108942 to base pair number 2108960.

d) Search in NCBI chromosome (bacteria Taxid: 2) database:

A total of 41 blast hits resulted when using SEQ ID NO:2 (from base pair number 2108819 to 2108960, GenBank accession number BA000033) as the query sequence to align NCBI chromosome (bacteria Taxid: 2) database under the least stringent condition (somewhat similar) (Table 1). Excluding the homology in the three S. aureus strains, the distribution of the rest of 38 blast hits aligned with DNA sequences that are random and short. Among them, only 3 hits have its 3′ end extended to the region corresponding to SEQ ID NO:2 base pair number 2108952. These 3 hits include two genes in Leptospira interrogans genome (GenBank accession number NC_{—}004342.1 and NC_{—}005823.1) with 82% homology, spanning the region of SEQ ID NO:2 from base pair number 2108914 to base pair number 2108952, and one gene in Clostridium botulinum (GenBank accession number NC_{—}010723.1) with 87% homology, spanning the region of SEQ ID NO:2 from base pair number 2108920 to base pair number 2108950. The most non-homologous region within SEQ ID NO:2 located at a region from base pair number 2108952 to base pair number 2108960.

e) Search in NCBI chromosome (Staphylococcus Taxid: 1279) database:

A total of 84 blast hits resulted when using SEQ ID NO:2 (from base pair number 2108819 to 2108960, GenBank accession number BA000033) as the query sequence to align NCBI chromosome (Staphylococcus Taxid: 1279) database under the least stringent condition (somewhat similar) (Table 1). Excluding the homology in the three S. aureus strains, the distribution of the rest of 81 blast hits clustered mostly within two regions of SEQ ID NO:2—from base pair number 2108835 to base pair number 2108863 and from base pair number 2108870 to base pair number 2108938. These two regions aligned with various S. aureus strains including S. aureus MSSA476 (GenBank accession number NC_{—}002953.3), S. aureus MW2 (GenBank accession number NC_{—}003923.1), S. aureus MRSA252 (GenBank accession number NC_{—}002952.2), S. aureus USA300_TCH1516 (GenBank accession number NC_{—}010079.1), S. aureus Mu3 (GenBank accession number NC_{—}009782.1), S. aureus Newman (GenBank accession number NC_{—}009641.1), S. aureus JH1 (GenBank accession number NC_{—}009632.1), S. aureus JH9 (GenBank accession number NC_{—}009487.1), S. aureus NCTC 8325(GenBank accession number NC_{—}007795.1), S. aureus USA300_FPR3757 (GenBank accession number NC_{—}007793.1), S. aureus N315 (GenBank accession number NC_{—}002745.2), S. aureus Mu50 (GenBank accession number NC_{—}002758.2), and S. aureus COL (GenBank accession number NC_{—}002951.2).

The alignment pick up one Staphylococcus saprophyticus ATCC15305 gene (GenBank accession number NC_{—}007350.1) with 88% homology, spanning the region of SEQ ID NO:2 from base pair number 2108875 to base pair number 2108899.

Three sequences in Staphylococcus haemolyticus JCSC1435 (GenBank accession number NC_{—}007168.1) are also aligned with SEQ ID NO:2. These three sequences correspond to SEQ ID NO:2 from base pair number 2108886 to base pair number 2108904 (94% homology); from base pair number 2108914 to base pair number 2108938 (88% homology); and from base pair number 2108911 to base pair number 2108929 (94% homology).

Four sequences in Staphylococcus epidermidis ATCC 12228 (GenBank accession number NC_{—}004461.1) are also aligned with SEQ ID NO:2. These four sequences correspond to SEQ ID NO:2 from base pair number 2108837 to base pair number 2108853 (100% homology), from base pair number 2108898 to base pair number 2108914 (100% homology), from base pair number 2108830 to base pair number 2108850 (90% homology), and from base pair number 2108835 to base pair number 2108884 (74% homology).

The most non-homologous region within SEQ ID NO:2 located at two regions—from base pair number 2108861 to base pair number 2108869 and from base pair number 2108939 to base pair number 2108960.

FIG. 1 Step 30: designing primer sets for amplifying specific agr gene sequence in CA-MRSA MW2 or CA-MRSA USA300 strains

a) primer set to amplify SEQ ID NO:1:

Because SEQ ID NO:1 is unique with no significant similarity found in all GenBank search performed, the following primer set was designed to amplified this DNA fragment:

(SEQ ID NO: 5)

forward primer 5′TTTTTATAATAATTTTTCTC3′

(SEQ ID NO: 6)

reverse primer 5′CTAAAAATACAGAATATAGT3′

b) primer set to amplify SEQ ID NO:2:

Based on FIG. 1 Step 20 1), 2) and 3), the following primer set was selected for amplifying SEQ ID NO:2 at the same time avoiding co-amplifying genes in human, bacteria or Staphylococcus sp. other then S. aureus strains:

(SEQ ID NO: 7)

forward primer 5′TTTATTAGTGGAATAAGAT3′

(SEQ ID NO: 8)

reverse primer 5′TTGTTATAACAACTAGTGA3′

c) primer set to amplify SEQ ID NO:3:

Because SEQ ID NO:3 is unique with no significant similarity found in all GenBank search performed, the following primer set was designed to amplified this 65 base pair DNA fragment:

(SEQ ID NO: 9)

forward primer 5′ATTTATACTTTTACCTTTA3′

(SEQ ID NO: 10)

reverse primer 5′TTAAAATAATCATAATTAAA3′

d) primer set to amplify SEQ ID NO:4:

Because SEQ ID NO:4 is unique with no significant similarity found in all GenBank search performed, the following primer set was designed to amplified this 66 base pair DNA fragment:

(SEQ ID NO: 11)

forward primer 5′TATTCTTTTATTTTTATTGGT3′

(SEQ ID NO: 12)

reverse primer 5′AAATTGAGAAATAACAAATGTT3′

Synthesis of these oligonucleotides can easily be done by commercial companies, such as Integrated DNA technologies (Coralville, Iowa).

FIG. 1 Step 40: labeling the agr primer sets at 5′ end with a fluorescent reporter attached methylisocytosine (iso-dC)

A newly developed Plexor system (Promega Corporation, Madison, Wis.) is applied to label a primer at 5′ end with a modified nucleotide (iso-dC) and a fluorophore. The selection of a fluorophore attached to each of the primer is based on the combination of the multiplex PCR amplification desired, as well as the detection capabilities of the real-time instrument used. The instruments currently available, with the compatibility to install the Plexor™ Analysis Software to visualize amplification data, plot standard curves and calculate DNA concentrations of unknowns, include ABI PRISM® 7000 and 7700 sequence detection systems, Applied Biosystems 7300, 7500 and 7900HT real time PCR system, Roche LightCycler® 1.0 and 2.0 instruments, Bio-Rad iCycler® thermal cycler, MJ Research DNA Engine Opticon® 2 fluorescence detection system, Cepheid SmartCycler® system and the Stratagene real-time PCR systems.

For example, if the available instrument is a Applied Biosystems 7500 real time PCR system (Forster City, Calif.) with 520 nm, 550 nm, 580 nm, 610 nm, and 650 nm filters installed, then the primers can be labeled in the order of use as FAM™, HEX™, Cal Fluor® Red 610, and Cy5™. The labeled primer can be synthesized by commercial companies such as Promega or Bioresearch Technologies.

To amplify and detect SEQ ID NO:1 and SEQ ID NO:2 for CA-MRSA MW2 strain, one can, for example, choose to label primers SEQ ID NO:5 and SEQ ID NO:7 with FAM™ and HEX™, respectively. Then, the remaining two filter channels are reserved for the multiplex PCR amplification for pvl and spa genes in one PCR reaction mixture.

Alternatively, to amplify and detect SEQ ID NO:3 and SEQ ID NO:4 for CA-MRSA USA300 strain in a separate reaction mixture, one can, for example, choose to label primers SEQ ID NO:9 and SEQ ID NO:11 with FAM™ and HEX™, respectively. Then, the remaining two filter channels are reserved for the multiplex PCR amplification for pvl and spa genes in one PCR reaction mixture.

A PCR reaction mixture can also be packaged into a combination of detecting CA-MRSA MW2 and USA300 strain in one tube by labeling SEQ ID NO:5 (for detecting SEQ ID NO:1 in MW2 strain) and SEQ ID NO:9 (for detecting SEQ ID NO:3 in USA300 strain) with FAM™ and HEX™, respectively. The remaining two filter channels are reserved for the multiplex PCR amplification for pvl and spa genes in one PCR reaction mixture.

Alternatively, if the purpose is only to type the agr gene (in the situation that the S. aureus status is already confirmed), then the combination of labeling SEQ ID NO:5 (for detecting SEQ ID NO:1 in CA-MRSA MW2 strain) and SEQ ID NO:7 (for detecting SEQ ID NO:2 in CA-MRSA MW2 strain) with FAM™ and HEX™, respectively, and SEQ ID NO:9 (for detecting SEQ ID NO:3 in CA-MRSA USA300 strain) and SEQ ID NO:11 (for detecting SEQ ID NO:4 in CA-MRSA USA300 strain) with Cal Fluor® Red 610 and Cy5™, respectively, can be packaged in one multiplex PCR mixture.

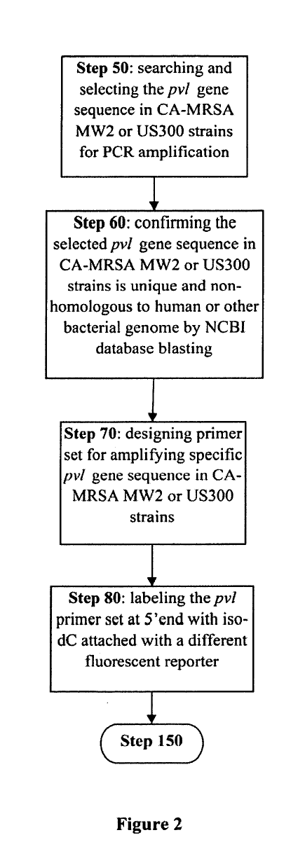

In the second dimension, this invention provides additional tool to amplify a partial pvl gene sequence in CA-MRSA MW2 or USA300 strains. The designing and labeling of the proper primer sequence to amplify pvl gene sequence facilitate the identification of virulent CA-MRSA. A flow chart of steps that describe the invention of proper pvl gene and primer sequences is depicted in FIG. 2. Detailed description of FIG. 2 is presented by steps as follows.

FIG. 2 Step 50: Searching and selecting the pvl gene sequences in CA-MRSA MW2 or USA300 strains for PCR amplification

Based on the published sequences of pvl genes (GenBank accession number X72700, S. aureus ATCC 49775), a blast 2 search was performed to align corresponding pvl sequences in CA-MRSA MW2 and USA300. The result showed that pvl sequence in CA-MRSA MW2 spans the region from base pair number 1918488 to base pair number 1920484 (GenBank accession number BA000033), and in CA-MRSA USA300, it spans the region from base pair number 1955328 to base pair number 1957326 (GenBank accession number NC_{—}007793). The pvl sequence in CA-MRSA MW2 and USA300 shares a 99% homology. A region in CA-MRSA MW2 pvl sequence spanning base pair number 1919380 to base pair number 1919820 (GenBank accession number BA000033) was selected for PCR amplification and primer design.

FIG. 2 Step 60: confirming the selected pvl gene sequence in CA-MRSA MW2 or USA300 strains is unique and non-homologous to human or other bacterial genome by NCBI database blasting

To avoid co-amplification of human genome and other possible contaminants, a region in CA-MRSA MW2 pvl sequence spanning base pair number 1919380 to base pair number 1919820 (GenBank accession number BA000033) was used as a query sequence in a local alignment to search for the homology within several databases in NCBI.

a) Search in human genome database:

A total of 96 blast hits resulted when using this CA-MRSA MW2 pvl sequence (from base pair number 1919380 to base pair number 1919820, GenBank accession number BA000033) as the query sequence to align human genome database under the least stringent condition (somewhat similar). However, there is no homology from base pair 1919380 to base pair number 1919429 and from base pair 1919740 to base pair number 1919820 (GenBank accession number BA000033).

b) Search in patent sequence database:

A total of 102 blast hits resulted when using this CA-MRSA MW2 pvl sequence (from base pair number 1919380 to base pair number 1919820, GenBank accession number BA000033) as the query sequence to align patent sequence database under the least stringent condition (somewhat similar). The result shows that most of these 102 blast hits are S. aureus pvl sequence derived from various strains. However, none of these patented sequences are primer sequences.

c) Search in NCBI chromosome database:

A total of 151 blast hits resulted when using this CA-MRSA MW2 pvl sequence (from base pair number 1919380 to 1919820, GenBank accession number BA000033) as the query sequence to align NCBI chromosome database under the least stringent condition (somewhat similar). The result shows that a total of 13 blast hits are S. aureus pvl sequence from various S. aureus strains which spans the whole region with the homology ranging from 100% to 98%. These S. aureus strains include S. aureus USA300_TCH1516 (GenBank accession number NC_{—}010079.1), S. aureus Newman (GenBank accession number NC_{—}009641.1), S. aureus NCTC 8325(GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), and. S. aureus RF122 (GenBank accession number NC_{—}007622.1).

The rest of the blast hits aligned with much shorter DNA regions with various organisms. However, there is no homology aligned to CA-MRSA MW2 pvl sequence from base pair number1919380 to base pair number 1919395 and from base pair base pair number 1919749 to base pair number 1919820 (GenBank accession number BA000033).

d) Search in NCBI chromosome (bacteria Taxid: 2) database:

A total of 87 blast hits resulted when using this CA-MRSA MW2 pvl sequence (from base pair number 1919380 to base pair number 1919820, GenBank accession number BA000033) as the query sequence to align NCBI chromosome (bacteria Taxid: 2) database under the least stringent condition (somewhat similar). The result also shows that a total of 13 blast hits are S. aureus pvl sequence from various S. aureus strains which spans the whole region with the homology ranging from 100% to 98%. These S. aureus strains include S. aureus USA300_TCH1516 (GenBank accession number NC_{—}010079.1), S. aureus Newman (GenBank accession number NC_{—}009641.1), S. aureus NCTC 8325(GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), and. S. aureus RF122 (GenBank accession number NC_{—}007622.1).

The rest of the blast hits aligned with much shorter DNA regions with various organisms. Except for one hit, there is no homology aligned to CA-MRSA MW2 pvl sequence from base pair number 1919380 to base pair number 1919404 and from base pair number 1919786 to base pair number 1919820 (GenBank accession number BA000033). The only one exception is a DNA sequence encoded a putative type II restriction enzyme Sau3AI by Bifidobacterium adolescentis ATCC 15703 (GenBank accession number NC_{—}008618.1) which shares 84% homology with CA-MRSA MW2 pvl sequence from base pair number 1919391 to base pair number 1919423 (GenBank accession number BA000033). B. adolescentis is not a human pathogen, and is unlikely to be contaminated with human clinical samples.

e) Search in NCBI chromosome (Staphylococcus Taxid: 1279) database:

A total of 131 blast hits resulted when using CA-MRSA MW2 pvl sequence (from base pair number 1919380 to base pair number 1919820, GenBank accession number BA000033) as the query sequence to align NCBI chromosome (Staphylococcus Taxid: 1279) database under the least stringent condition (somewhat similar).

The result also shows that a total of 13 blast hits are S. aureus pvl sequence from various S. aureus strains which spans the whole region with the homology ranging from 100% to 98%. These S. aureus strains include S. aureus USA300_TCH1516 (GenBank accession number NC_{—}010079.1), S. aureus Newman (GenBank accession number NC_{—}009641.1), S. aureus NCTC 8325 (GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), and. S. aureus RF122 (GenBank accession number NC_{—}007622.1).

The rest of the blast hits aligned with much shorter DNA regions in several coagulase negative Staphylococcus including S. haemolyticus JCSC1435 (GenBank accession number NC_{—}007168.1), S. saprophyticus ATCC15305 (GenBank accession number NC_{—}007350.1), and S. epidermidis RP62A (GenBank accession number NC_{—}002976.3). However, none of the homologous regions aligned with CA-MRSA MW2 pvl sequence from base pair number 1919380 to base pair number 1919407 and from base pair number 1919560 to base pair number 1919820 (GenBank accession number BA000033).

FIG. 2 Step 70: designing primer set for amplifying specific pvl gene sequence in CA-MRSA MW2 or USA300 strains.

Based on the FIG. 2 Step 60 a), b), c), d) and e), the following primer set was selected for amplifying pvl gene at the same time avoiding co-amplifying human and other bacterial sequences:

(SEQ ID NO: 13)

forward primer 5′CAGGTGTGATATGTTGAGCT3′

(SEQ ID NO: 14)

reverse primer 5′TATTTATTCGTACAAAGTCCA3′

Synthesis of these oligonucleotides can easily be done by commercial companies, such as Integrated DNA technologies (Coralville, Iowa).

FIG. 2 Step 80: labeling the pvl primer set at 5′end with a fluorescent reporter attached iso-dC

A newly developed Plexor system (Promega Corporation, Madison, Wis.) as described in FIG. 1 Step 40 is applied to label a primer at 5′ end with a modified nucleotide (iso-dC) and a fluorophore. The selection of a fluorophore attached to the primer is based on the combination of the multiplex PCR amplification desired, as well as the detection capabilities of the real-time instrument used as described in FIG. 1 Step 40.

For example, as described in FIG. 1 Step 40, if the available instrument is a Applied Biosystems 7500 real time PCR system (Forster City, Calif.) with 520 nm, 550 nm, 580 nm, 610 nm, and 650 nm filters installed, then the primers can be labeled in the order of use as FAM™, HEX™, Cal Fluor® Red 610, and Cy5™. The labeled primer can be synthesized by commercial companies such as Promega (Madison, Wis.).

As described in FIG. 1 Step 40, FAM™ and HEX™ may be used to label primers SEQ ID NO:5 and SEQ ID NO:7 to amplify and detect SEQ ID NO:1 and SEQ ID NO:2 for CA-MRSA MW2 strain. In this case, one can choose to label SEQ ID NO:13 with Cal Fluor® Red 610 for the detection of pvl gene as an example.

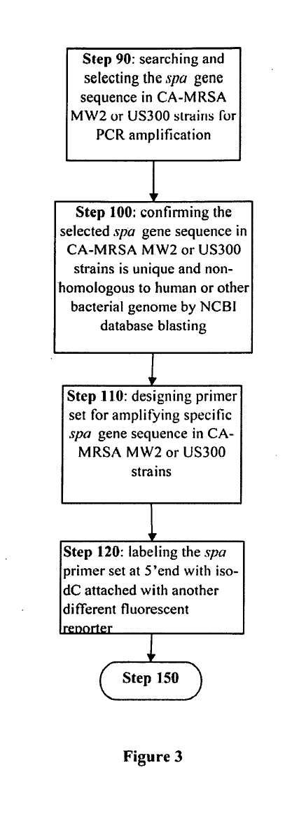

In the third dimension, this invention provides additional tool to amplify a partial spa gene sequence in CA-MRSA MW2 or USA300 strains. The designing and labeling of the proper primer sequence to amplify spa gene sequence discriminates staphylococci other than S. aureus strains. A flow chart of steps that describe the invention of proper spa gene and primer sequences is depicted in FIG. 3. Detailed description of FIG. 3 is presented by steps as follows.

FIG. 3 Step 90: searching and selecting the spa gene sequences in CA-MRSA MW2 or USA300 strains for PCR amplification

A blast 2 search was performed, based on the published sequences of spa gene (GenBank accession number J01786, S. aureus strain 8325-4, Uhlen et al., 1984, JBC 259:1695-1702), to align corresponding spa sequences in CA-MRSA MW2 and USA300. A short DNA region, spanning spa gene sequence in CA-MRSA MW2 from base pair number 99612 to base pair number 99792 (GenBank accession number BA000033) was selected for PCR amplification and primer design. The spa sequences in CA-MRSA MW2 and USA300 share a 100% homology in this region.

FIG. 3 Step 100: confirming the selected spa gene sequence in CA-MRSA MW2 or USA300 strains is unique and non-homologous to human or other bacterial genome by NCBI database blasting

To avoid co-amplification of human genome and other possible contaminants, a region in CA-MRSA MW2 spa sequence spanning base pair number 99652 to base pair number 99792 (GenBank accession number BA000033) was used as a query sequence in a local alignment to search for the homology within several databases in NCBI.

a) Search in human genome database:

A total of 22 blast hits resulted when using this CA-MRSA MW2 spa sequence (from base pair number 99652 to base pair number 99792, GenBank accession number BA000033) as the query sequence to align human genome database under the least stringent condition (somewhat similar). However, there is no homology from base pair number 99652 to base pair number 99664 and from base pair 99750 to base pair number 99767 (GenBank accession number BA000033).

b) Search in patent sequence database:

A total of 100 blast hits resulted when using this CA-MRSA MW2 spa sequence (from base pair number 99652 to base pair number 99792, GenBank accession number BA000033) as the query sequence to align patent sequence database under the least stringent condition (somewhat similar). The result shows that many of these 100 blast hits are S. aureus spa sequence, however, none of these patented sequences are primer sequences.

c) Search in NCBI chromosome database:

A total of 54 blast hits resulted when using this CA-MRSA MW2 spa sequence (from base pair number 99652 to base pair number 99792, GenBank accession number BA000033) as the query sequence to align NCBI chromosome database under the least stringent condition (somewhat similar). The result shows that a total of 14 blast hits are S. aureus spa sequence from various S. aureus strains which spans the whole region with the homology ranging from 100% to 90%. These S. aureus strains include S. aureus USA300_TCH1516 (GenBank accession number NC_{—}010079.1), S. aureus Newman (GenBank accession number NC_{—}009641.1), S. aureus NCTC 8325 (GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), S. aureus MRSA252 (GenBank accession number NC_{—}002952.2), and S. aureus RF122 (GenBank accession number NC_{—}007622.1).

The rest of the blast hits aligned with much shorter DNA regions with various organisms. These DNA regions are too short and unlikely to be amplified when CA-MRSA MW2 spa sequence from base pair number 99652 to base pair number 99664 and from base pair number 99750 to base pair number 99767 (GenBank accession number BA000033) are used as primer sets for PCR.

d) Search in NCBI chromosome (bacteria Taxid: 2) database:

A total of 70 blast hits resulted when using this CA-MRSA MW2 spa sequence (from base pair number 99651 to base pair number 99792, GenBank accession number BA000033) as the query sequence to align NCBI chromosome (bacteria Taxid: 2) database under the least stringent condition (somewhat similar). The result also shows that a total of 14 blast hits are S. aureus spa sequence from various S. aureus strains which spans the whole region with the homology ranging from 100% to 99%. These S. aureus strains include S. aureus USA300_TCH1516 (GenBank accession number NC_{—}010079.1), S. aureus Newman (GenBank accession number NC_{—}009641.1), S. aureus NCTC 8325(GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), S. aureus MRSA252 (GenBank accession number NC_{—}002952.2) and S. aureus RF122 (GenBank accession number NC_{—}007622.1).

The rest of the blast hits aligned with much shorter DNA regions with various organisms. These DNA regions are too short and unlikely to be amplified when CA-MRSA MW2 spa sequence from base pair number 99652 to base pair number 99664 and from base pair 99750 to base pair number 99767 (GenBank accession number BA000033) are used as primer sets for PCR.

e) Search in NCBI chromosome (Staphylococcus Taxid: 1279) database:

A total of 74 blast hits resulted when using CA-MRSA MW2 spa sequence (from base pair number 99651 to base pair number 99792, GenBank accession number BA000033) as the query sequence to align NCBI chromosome (Staphylococcus Taxid: 1279) database under the least stringent condition (somewhat similar).

The result also shows that a total of 14 blast hits are S. aureus spa sequence from various S. aureus strains which spans the whole region with the homology ranging from 100% to 99%. These S. aureus strains include S. aureus USA300_TCH1516 (GenBank accession number NC_{—}010079.1), S. aureus Newman (GenBank accession number NC_{—}009641.1), S. aureus NCTC 8325 (GenBank accession number NC_{—}007795.1), S. aureus USA300 (GenBank accession number NC_{—}007793.1), S. aureus COL (GenBank accession number NC_{—}002951.2), S. aureus MSSA476 (GenBank accession number NC_{—}002953.3), S. aureus MW2 (GenBank accession number NC_{—}003923.1), S. aureus Mu3 (GenBank accession number NC_{—}009782.1), S. aureus JH1 (GenBank accession number NC_{—}009632.1), S. aureus JH9 (GenBank accession number NC_{—}009487.1), S. aureus N315 (GenBank accession number NC_{—}002745.2), S. aureus Mu50 (GenBank accession number NC_{—}002758.2), S. aureus MRSA252 (GenBank accession number NC_{—}002952.2) and S. aureus RF122 (GenBank accession number NC_{—}007622.1).

The rest of the blast hits aligned with much shorter DNA regions in several coagulase negative Staphylococcus including S. haemolyticus JCSC1435 (GenBank accession number NC_{—}007168.1), S. saprophyticus ATCC15305 (GenBank accession number NC_{—}007350.1), S. epidermidis ATCC12228 (GenBank accession number NC_{—}004461.1) and S. epidermidis RP62A (GenBank accession number NC_{—}002976.3). However, these DNA regions are too short and unlikely to be amplified when MW2 spa sequence from base pair number 99652 to base pair number 99664 and from base pair number 99750 to base pair number 99767 (GenBank accession number BA000033) are used as primer sets for PCR.

FIG. 3 Step 110: designing primer sets for amplifying specific spa gene sequence in CA-MRSA MW2 or USA300 strains

Based on the FIG. 3 Step 100 a), b), c), d), and e), the following primer set was selected for amplifying spa gene at the same time avoiding co-amplifying human and other bacterial sequences:

(SEQ ID NO: 15)

forward primer 5′TCAACAACAAGTTCTTGACC3′

(SEQ ID NO: 16)

reverse primer 5′ACAGTAAATGACATTGCAAAA3′

Synthesis of these oligonucleotides can easily be done by commercial companies, such as Integrated DNA technologies (Coralville, Iowa).

FIG. 3 Step 120: labeling the spa primer set at 5′end with a fluorescent reporter attached iso-dC

A newly developed Plexor system (Promega Corporation, Madison, Wis.) as described in FIG. 1 Step 40 and FIG. 2 Step 80 is applied to label a primer at 5′ end with a modified nucleotide (iso-dC) and a fluorophore. The selection of a fluorophore attached to the primer is based on the combination of the multiplex PCR amplification desired, as well as the detection capabilities of the real-time instrument used as described in FIG. 1 Step 40 and FIG. 2 Step 80.

For example, as described in FIG. 1 Step 40 and FIG. 2 Step 80, if the available instrument is a Applied Biosystems 7500 real time PCR system (Forster City, Calif.) with 520 nm, 550 nm, 580 nm, 610 nm, and 650 nm filters installed, then the primers can be labeled in the order of use as FAM™, HEX™, Cal Fluor® Red 610, and Cy5™. The labeled primer can be synthesized by commercial companies such as Promega (Madison, Wis.).

As described in FIG. 1 Step 40 and FIG. 2 Step 80, FAM™ HEX™ and Cal Fluor® Red 610 may be used to label primers SEQ ID NO:5, SEQ ID NO:7 and SEQ ID NO:13, respectively, to amplify and detect SEQ ID NO:1 and SEQ ID NO:2 for CA-MRSA MW2 strain and pvl gene. In this case, one can choose to label SEQ ID NO:15 with Cy5™ and package them in one multiplex PCR amplification mixture for the detection of spa gene.

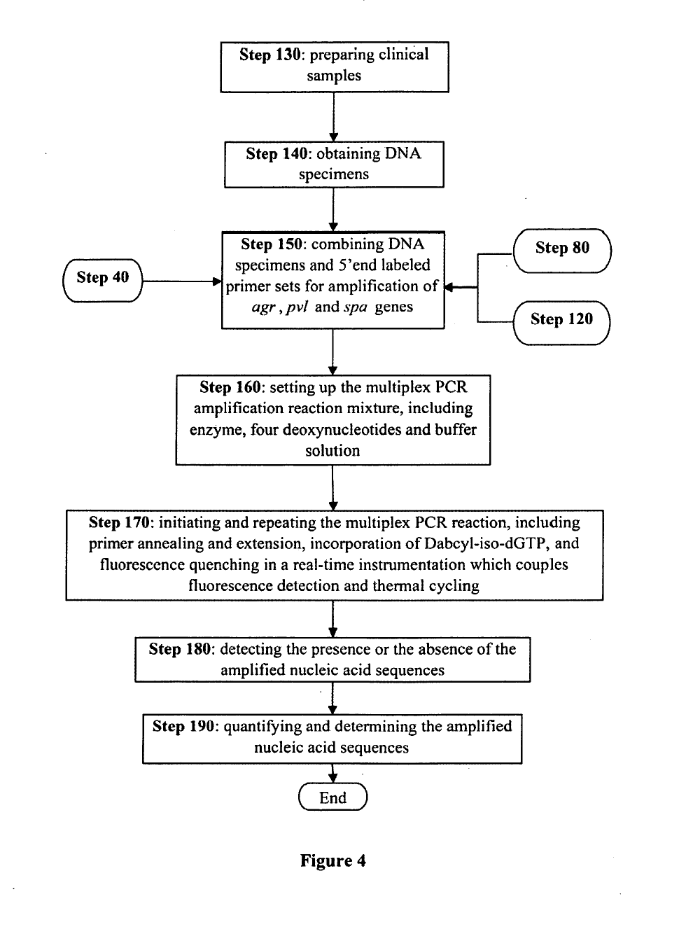

In the fourth dimension, this invention provides methods in identifying CA-MRSA MW2 or USA300 strains in human clinical samples. Using proper primer sequences provided and described in FIGS. 1, 2, and 3 in multiplex PCR amplification reactions, the target DNA sequences that represent agr, pvl, and spa genes in CA-MRSA MW2 or USA300 strains can be identified. A flow chart of steps that describe methods to identify these virulence genes is depicted in FIG. 4. Detailed description of FIG. 4 is presented by steps as follows.

FIG. 4 Step 130: preparing clinical samples

Clinical samples suitable for this invention can be 1) specimens from skin pustules, ears, conjunctiva, draining tracts or wounds, soft tissue infections, or the reproductive tract that were obtained by swabs; 2) whole blood, buffy coat, serum or plasma; 3) cerebrospinal and synovial fluid; 4) specimens from devises or catheter tubing implanted in human bodies; 5) environmental samples and 6) isolated colonies. Depending on the quantity of the material, these samples may or may not require to be inoculated onto both general purpose (such as trypticase/tryptic soy agar with 5% sheep blood, Difco, Detroit, Mich.) and selective media (mannitol salt agar) to maximize bacterial recovery and single colony isolation. After incubating the agar plates 37° C. overnight, 3-5 colonies can be collected and suspended in 100 μl Tris-EDTA pH 7.5 buffers. The samples are ready for DNA extraction.

Alternatively, for collecting clinical samples from skin lesions with pus and abscesses, a sterile curette can be used to collect the samples and then inserted into a sterile tubes containing 1 ml tryptic soy broth (Difco, Detroit, Mich.). Samples can then be vortexed and the aliquot is ready for DNA extraction.

FIG. 4 Step 140: obtaining DNA specimens

There are commercial kits available for extraction and isolation for bacterial DNA, such as Qiagen QIAamp blood and tissue kit (Qiagen, Gaithersburg, Md.), Roche High Pure PCR Template Preparation Kit (Roche, Alameda, Calif.) and NucPrep DNA isolation kit (Applied Biosystems, Foster City, Calif.). For large numbers of samples, a 96-well deep-well plate on the ABI PRISM™6100 Nucleic Acid Prep Station (Applied Biosystems, Foster City, Calif.) with the NucPrep DNA isolation kit (Applied Biosystems, Foster City, Calif.) is ideal for DNA specimens' preparation.

For example, DNA from clinical isolates, as well as standard CA-MRSA MW2 or USA300 strains, is extracted with the NucPrep DNA isolation kit according to the manufacturer's instructions. In brief, lysozyme and lysostaphin is added to a 96-well deep-well plate containing bacteria suspended in 100 μl Tris-EDTA buffer. The μlate is incubated at 37° C. for 60 min, followed by the addition of proteinase K and incubation at 70° C. for 30 min. The samples are further processed according to the manufacturer's instructions. The bacterial DNA is eluted in a total volume of 200 μl.

Alternatively, 100 μl of 5M guanidine HCl is added to each well of a 96-well deep-well plate containing bacteria suspended in 100 μl Tris-EDTA buffer and the plate is vortexed briefly. The plate was then sonicated in an ultrasonicator bath for 15 min to lyse the bacteria. This is followed by the addition of 1 ml of NucPrep DNA purification solution and brief vortexing. The sample is further processed according to the manufacturer's instructions. The bacterial DNA is eluted in a total volume of 200 μl.

FIG. 4 Step 150: combining DNA specimen and 5′end labeled primer sets for amplification of agr, pvl and spa genes

Clinical DNA specimens from FIG. 4 Step 140 are used as DNA templates for multiplex PCR amplification on a 96-well real-time instrument, for example, Applied Biosystems 7500 (Foster City, Calif.). In general, 5-10 μl template DNA and 0.1-0.2 μM of the each forward and reverse primer for detecting agr, pvl, and spa genes is used in a Plexor™ One-Step multiplex PCR system (Promega, Madison, Wis.). One in each set of the primer is labeled with iso-dC and a fluorophore as described in FIG. 1 Step 40, FIG. 2 Step 80 and FIG. 3 Step 120. The selection of a fluorophore attached to the primer is based on the combination of the multiplex PCR amplification desired, as well as the detection capabilities of the real-time instrument used as described in FIG. 1 Step 40, FIG. 2 Step 80, and FIG. 3 Step 120.

FIG. 4 Step 160: setting up the multiplex PCR amplification reaction mixture, including enzyme, four deoxynucleotides and buffer solution

Basically, setting up the multiplex PCR amplification reaction mixture can be performed by following the manufacture's instruction (Promega, Madison, Wis.). Plexor™ One-Step multiplex PCR reaction system (Promega, Madison, Wis.) includes all the necessary components, such as DNA polymerase, dabcyl-iso-dGTP (as a fluorescence quencher), four deoxynucleotides, buffer and salt solution and a primer-dimer inhibitor. Proper amount of the Plexor™ One-Step multiplex PCR reaction mixture is add to each of the 96-well described in FIG. 4 Step 150.

FIG. 4 Step 170: initiating and repeating the multiplex PCR amplification reaction, including primer annealing and extension, incorporation of dabcyl-iso-dGTP, and fluorescence quenching in a real-time instrumentation which couples fluorescence detection and thermal cycling

A real-time PCR instrument, for example, the Applied Biosystems 7500 (Foster City, Calif.) with the installation of the Plexor™ Analysis Software (Promega, Madison, Wis.) is programmed to initiate and repeat the multiplex PCR amplification reaction. Cycles of amplification is performed.

FIG. 4 Step 180: measuring the fluorescence change at every cycle

Plexor™ technology takes advantage of the highly specific interaction between two modified nucleotides for quantitative PCR analysis. These two novel bases, iso-dG and 5′-methylisocytosine (iso-dC), form a unique base pair when incorporated in double-stranded DNA and pair only with each other (Promega, Madison, Wis.). During the amplification reaction, only dabcyl-iso-dGTP can be incorporated at the position complementary to the iso-dC residue. Incorporation of the dabcyl-iso-dGTP in close proximity to the fluorescent label effectively quenches the fluorescent signal. The accumulation of amplification product results in a reduction in fluorescence that is proportional to the quantity of the input DNA template. The use of the Plexor™ Analysis Software enables the visualization of amplification data in real-time by measuring the change of signal (in Relative Fluorescent Unites, RFU) at every cycle.

For example, as described in FIG. 1 Step 40, FIG. 2 Step 80 and FIG. 3 Step 120, assuming the available instrument is an Applied Biosystems 7500 real time PCR system (Forster City, Calif.) with 520 nm, 550 nm, 580 nm, 610 nm, and 650 nm filters installed, and the primers are labeled in the order of use as FAM™, HEX™, Cal Fluor® Red 610, and Cy5™. One can use FAM™ HEX™, Cal Fluor® Red 610 and Cy5™ to label primers SEQ ID NO:5, SEQ ID NO:7, SEQ ID NO:13, and SEQ ID NO:15, respectively, to amplify and detect SEQ ID NO:1, SEQ ID NO:2, pvl gene and spa gene, respectively, for identifying CA-MRSA MW2 strain in one multiple PCR amplification in real-time.

For another example, as described in FIG. 1 Step 40, FIG. 2 Step 80 and FIG. 3 Step 120, assuming the available instrument is an Applied Biosystems 7500 real time PCR system (Forster City, Calif.) with 520 nm, 550 nm, 580 nm, 610 nm, and 650 nm filters installed, and the primers are labeled in the order of use as FAM™, HEX™, Cal Fluor® Red 610, and Cy5™. One can use FAM™ HEX™, Cal Fluor® Red 610 and Cy5™ to label primers SEQ ID NO:9, SEQ ID NO:11, SEQ ID NO:13, and SEQ ID NO:15, respectively, to amplify and detect SEQ ID NO:3, SEQ ID NO:4, pvl gene and spa gene, respectively, for identifying CA-MRSA USA300 strain in one multiple PCR amplification in real-time.

FIG. 4 Step 190: quantifying and determining the amplified nucleic acid sequence

Amplification results from Plexor™ reactions present a characteristic three-phase curve. Results obtained during the exponential phase of amplification give the best estimate of the amount of starting material. The amplification curve of each product can thus be generated using the values from a dilution series of a sample with a known DNA quantity.

In Plexor™ reactions, the quenching of the fluorescent label by dabcyl is a reversible process. When the product is double-stranded, the dabcyl and fluorescent label are in close proximity and fluorescence is quenched. Denaturing the double-stranded product separates the label and quencher, resulting in an increase in fluorescent signal. Thus, thermal melt curves can be generated and used to determine the melting temperature of each of the amplification product.

Using the example as described in FIG. 4 Step 180, when FAM™, HEX™, Cal Fluor® Red 610 and Cy5™ are used to label primers SEQ ID NO:5, SEQ ID NO:7, SEQ ID NO:13, and SEQ ID NO:15, respectively, to amplify and detect SEQ ID NO:1, SEQ ID NO:2, pvl gene and spa gene regions, respectively, amplification products in sizes of a) 54 base pair (portions of SEQ ID NO:1, GenBank accession number BA000033, from base pair number 2108971 to base pair number 2109024) labeled with FAM™, b) 115 base pair (portions of SEQ ID NO:2 plus extra primer sequence at 3′end of SEQ ID NO:2, GenBank accession number BA000033, from base pair number 2108856 to base pair number 2108970) labeled with HEX™, c) 169 base pair (combination of the above two DNA sequences, GenBank accession number BA000033, from base pair number 2108856 to base pair number 2109024) labeled with HEX™, d) 419 base pair (portions of pvl gene, GenBank accession number BA000033, from base pair number 1919366 to base pair number 1919784) labeled with Cal Fluor® Red 610, and e) 110 base pair (portions of spa gene, GenBank accession number BA000033, from base pair number 99683 to base pair number 99792) labeled with Cy5™. Each of the amplification products has a distinguishable melting curve due the differences of their size and nucleotide compositions. Clinical samples containing CA-MRSA MW2 strains will be showing positive amplification for all five above mentioned DNA amplification products. Alternatively, clinical specimens containing MSSA476, MRSA252, or agr-3ₛₐ strains will be showing positive amplification for the b) 115 base pair, d) 419 base pair, and e) 110 base pair as DNA amplification products. The all negative result indicates the absence of any S. aureus in the clinical specimens, while the presence of only d) 419 base pair and e) 110 base pair as DNA amplification products indicate the presence of S. aureus in the clinical specimens which may not be CA-MRSA MW2, MSSA476, MRSA252, or agr-3ₛₐ strains.

As for the identification of CA-MRSA USA300 strains in a separate PCR amplification reaction, when FAM™, HEX™, Cal Fluor® Red 610 and Cy5™ are used to label primers SEQ ID NO:9, SEQ ID NO:11, SEQ ID NO:13, and SEQ ID NO:15, respectively, to amplify and detect SEQ ID NO:3, SEQ ID NO:4, pvl gene and spa gene regions, respectively, amplification products in sizes of a) 65 base pair (SEQ ID NO:3, GenBank accession number NC_{—}007793.1, from base pair number 2147214 to base pair number 2147278) labeled with FAM™, b) 66 base pair (SEQ ID NO:4, GenBank accession number NC_{—}007793.1, from base pair number 2148216 to base pair number 2148281) labeled with HEX™, c) 1068 base pair (amplified by the primer set SEQ ID NO:9 and SEQ ID NO:12, GenBank accession number NC_{—}007793.1, from base pair number 2147214 to base pair number 2148281) labeled with FAM™, d) 419 base pair (portions of pvl gene, GenBank accession number NC_{—}007793.1, from base pair number 1956206 to base pair number 1956624) labeled with Cal Fluor® Red 610, and e) 110 base pair (portions of spa gene, GenBank accession number NC_{—}007793.1, from base pair number 128321 to base pair number 128430) labeled with Cy5™. Each of the amplification products has a distinguishable melting curve due the differences of their size and nucleotide compositions. Clinical specimens containing CA-MRSA USA300 strains will be showing positive amplification for at least a) 65 base pair, b) 66 base pair, d) 419 base pair, and e) 110 base pair DNA amplification products. The 1068 base pair DNA product may not be efficiently amplified due to its length. The all negative result indicates the absence of any S. aureus in the clinical specimens, while the presence of only d) 419 base pair and e) 110 base pair as DNA amplification products indicate the presence of S. aureus in the clinical specimens which may not be CA-MRSA USA300 strains.

Depending upon the availability of the fluorescent labeling and the compatibility of the detection instrument, this invention uses various combinations of the primer sequences with various labeling to achieve a multiplex PCR amplification for identifying the most pathogenic and virulent strains among S. aureus.

What is claimed is:

1. A method for detecting community-acquired methicillin-resistant Staphylococcus aureus (CA-MRSA) MW2 or USA300 strains from a DNA specimen, which comprises:
a) Preparing clinical samples;
b) Obtaining DNA specimens;
c) Combining said DNA specimens with a plurality of primers, wherein said primers comprises SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16;
d) Setting up a plurality of conditions for a multiplex PCR amplification reaction to get amplified nucleic acid sequences;
e) Initiating and repeating the multiplex PCR amplification reaction;
f) Detecting the presence or the absence of a plurality of amplified nucleic acid sequences; and
g) Quantifying and determining a plurality of amplified nucleic acid sequences.

2. The method of claim 1, wherein said clinical samples comprise human samples from skin lesions, wounds, soft tissue infections, blood, cerebrospinal fluid, synovial fluid and any other body part; samples from medical devices or tubes that are implanted or inserted in human bodies;
and environmental samples.

3. The method of claim 1, wherein said preparing clinical samples comprises suspending clinical samples in a liquid without enrichment; inoculating clinical samples onto agar plates for single colony isolation; or suspending clinical samples in a liquid with enrichment.

4. The method of claim 1, wherein said obtaining DNA specimens comprises treating said clinical samples with reagents selected from the group comprising of guanidine, detergent, protease, lysostaphin, achromopeptidase or any commercial available DNA extraction kit.

5. The method of claim 1, wherein said DNA specimens are combined with said primers comprising of SEQ ID NO:5, SEQ ID NO:6, SEQ ID NO:7, SEQ ID NO:8, SEQ ID NO:13, SEQ ID NO:14, SEQ ID NO:15 and SEQ ID NO:16.

6. The method of claim 5, wherein said primers SEQ ID NO:5, SEQ ID NO:7, SEQ ID NO:13 and SEQ ID NO:15 are further conjugated with methylisocytosine (iso-dC) to the 5′ end and then adding a different fluorophore to each primer adjacent to the iso-dC.

7. The method of claim 1, wherein said DNA specimens are combined with said primers comprising of SEQ ID NO:9, SEQ ID NO:10, SEQ ID NO:11, SEQ ID NO:12, SEQ ID NO:13, SEQ ID NO:14, SEQ ID NO:15 and SEQ ID NO:16.

8. The method of claim 7, wherein said primers SEQ ID NO:9, SEQ ID NO:11, SEQ ID NO:13 and SEQ ID NO:15 are further conjugated with methylisocytosine (iso-dC) to the 5′ end and then adding a different fluorophore to each primer adjacent to the iso-dC.

9. The method of claim 1, wherein said DNA specimens are combined with said primers comprising of SEQ ID NO:5, SEQ ID NO:6, SEQ ID NO:7, SEQ ID NO:8, SEQ ID NO:9, SEQ ID NO:10, SEQ ID NO:11 and SEQ ID NO:12.

10. The method of claim 9, wherein said primers SEQ ID NO:5, SEQ ID NO:7, SEQ ID NO:9 and SEQ ID NO:11 are further conjugated with methylisocytosine (iso-dC) to the 5′ end and then adding a different fluorophore to each primer adjacent to the iso-dC.

11. The method of claim 1, wherein said DNA specimens are combined with any combination of primer sets comprising of SEQ ID NO:5, SEQ ID NO:6, SEQ ID NO:7, SEQ ID NO:8, SEQ ID NO:9, SEQ ID NO:10, SEQ ID NO:11, SEQ ID NO:12, SEQ ID NO:13, SEQ ID NO:14, SEQ ID NO:15 or SEQ ID NO:16.

12. The method of claim 11, wherein one of the said primer in a primer set is further conjugated with methylisocytosine (iso-dC) to the 5′ end and then adding a plurality of fluorophore adjacent to the iso-dC in each of the chosen primer.

13. The method of claim 1, wherein said conditions for a multiplex PCR amplification reaction comprise a DNA polymerase, four nucleotide dATP, dCTP, dGTP, dTTP, buffer and salt solutions, dabcyl-iso-dGTP, a primer-dimer inhibitor, or any commercial available reagents.

14. The method of claim 1, wherein said initiating and repeating the multiplex PCR amplification reaction comprises the use of a real-time instrumentation which couples fluorescence detection and thermal cycling. The fluorescence detection step comprises the installation of proper computer software for detecting said fluorophore. The thermal cycling step comprises incorporating Dabcyl-iso-dGTP and fluorescence quenching, DNA denaturing, primer annealing and extension.

15. The method of claim 1, wherein said detecting the presence or the absence of a plurality of amplified nucleic acid sequences comprises measuring the fluorescence change of each said fluorophore at every cycle.

16. The method of claim 15, wherein said a plurality of amplified nucleic acid sequences comprises SEQ ID NO:1, SEQ ID NO:2, a partial pvl gene and a partial spa gene.

17. The method of claim 15, wherein said a plurality of amplified nucleic acid sequences comprises SEQ ID NO:3, SEQ ID NO:4, a partial pvl gene and a partial spa gene.

18. The method of claim 1, wherein said quantifying and determining a plurality of the amplified nucleic acid sequences comprises generating amplification curve and thermal melting curve for each said amplified nucleic acid sequence.

19. The method of claim 18, wherein said a plurality of amplified nucleic acid sequences comprises SEQ ID NO:1, SEQ ID NO:2, a partial pvl gene and a partial spa gene.

20. The method of claim 18, wherein said a plurality of amplified nucleic acid sequences comprises SEQ ID NO:3, SEQ ID NO:4, a partial pvl gene and a partial spa gene.


SEQUENCE LISTING (16 sequences)

SEQ ID NO: 1 - DNA, 64 bp, Staphylococcus aureus
  misc_feature: agr gene of CA-MRSA MW2
gttataacaatttttataataatttttctctattttaaaattagactatattctgtatttttag

SEQ ID NO: 2 - DNA, 142 bp, Staphylococcus aureus
  misc_feature: arg gene of CA-MRSA MW2
aattgttcaagtttcattaatgttctttatatctgcatttattagtggaataagatacaaaaaatcagattatatatacattattggaatagtgttatcttcagtatatttctttgacaaaatcggaagtatttcactagtt

SEQ ID NO: 3 - DNA, 65 bp, Staphylococcus aureus
  misc_feature: agr gene of CA-MRSA USA300
atttatacttttacctttagtaatagtaaattttcatattaactttttaattatgattattttaa

SEQ ID NO: 4 - DNA, 66 bp, Staphylococcus aureus
  misc_feature: agr gene of CA-MRSA USA300
tattcttttatttttattggtatcactatatttttaagtatattaacatttgttatttctcaattt

SEQ ID NO: 5 - DNA, 20 bp, Staphylococcus aureus
  misc_feature: forward primer specific for a target DNA sequence of the agr gene of CA-MRSA MW2
tttttataataatttttctc

SEQ ID NO: 6 - DNA, 20 bp, Staphylococcus aureus
  misc_feature: reverse primer specific for a target DNA sequence of the agr gene of CA-MRSA MW2
ctaaaaatacagaatatagt

SEQ ID NO: 7 - DNA, 19 bp, Staphylococcus aureus
  misc_feature: forward primer specific for a target DNA sequence of the agr gene of CA-MRSA MW2
tttattagtggaataagat

SEQ ID NO: 8 - DNA, 19 bp, Staphylococcus aureus
  misc_feature: reverse primer specific for a target DNA sequence of the agr gene of CA-MRSA MW2
ttgttataacaactagtga

SEQ ID NO: 9 - DNA, 19 bp, Staphylococcus aureus
  misc_feature: forward primer specific for a target DNA sequence of the agr gene of CA-MRSA USA300
atttatacttttaccttta

SEQ ID NO: 10 - DNA, 20 bp, Staphylococcus aureus
  misc_feature: reverse primer specific for a target DNA sequence of the agr gene of CA-MRSA USA300
ttaaaataatcataattaaa

SEQ ID NO: 11 - DNA, 21 bp, Staphylococcus aureus
  misc_feature: forward primer specific for a target DNA sequence of the agr gene of CA-MRSA USA300
tattcttttatttttattggt

SEQ ID NO: 12 - DNA, 22 bp, Staphylococcus aureus
  misc_feature: reverse primer specific for a target DNA sequence of the agr gene of CA-MRSA USA300
aaattgagaaataacaaatgtt

SEQ ID NO: 13 - DNA, 20 bp, Staphylococcus aureus
  misc_feature: forward primer specific for a target DNA sequence of the pvl gene of CA-MRSA
caggtgtgatatgttgagct

SEQ ID NO: 14 - DNA, 21 bp, Staphylococcus aureus
  misc_feature: reverse primer specific for a target DNA sequence of the pvl gene of CA-MRSA
tatttattcgtacaaagtcca

SEQ ID NO: 15 - DNA, 20 bp, Staphylococcus aureus
  misc_feature: forward primer specific for a target DNA sequence of the spa gene of CA-MRSA
tcaacaacaagttcttgacc

SEQ ID NO: 16 - DNA, 21 bp, Staphylococcus aureus
  misc_feature: reverse primer specific for a target DNA sequence of the spa gene of CA-MRSA
acagtaaatgacattgcaaaa